(19) United States
(12) Patent Application Publication
(10) Pub. No.: US 2024/0033240 A1
(43) Pub. Date: Feb. 1, 2024

(54) DUAL RESPONSIVE BRAIN TARGETED NANOPARTICLES FOR USE IN TREATMENT OF ALZHEIMER'S DISEASE

(71) Applicant: UNIVERSITY OF SOUTH CAROLINA, COLUMBIA, SC (US)

(72) Inventors: Peisheng Xu, Chapin, SC (US); Eleni Markoutsa, Cayce, SC (US)

(21) Appl. No.: 18/479,393

(22) Filed: Oct. 2, 2023

Related U.S. Application Data
(62) Division of application No. 15/164,994, filed on May 26, 2016.
(60) Provisional application No. 62/167,563, filed on May 28, 2015.

(51) Int. Cl. A61K 31/198; A61K 47/69; A61K 47/54; A61K 47/55

(52) U.S. Cl. CPC A61K 31/198; A61K 47/6933; A61K 47/6935; A61K 47/54; A61K 47/55

(57) ABSTRACT

The preparation of dual functionalized nanoparticles is generally provided along with their application. The dual functionalized nanoparticles provide dual targeting and can effectively pass the blood brain barrier and target brain tissue. The dual targeted and dual responsive nanoparticles are functionalized to include at least two different ligands that are capable of transport across the blood brain barrier. The nanoparticles can be prepared from polymeric materials that can be biocompatible, provide long circulation life in a body, and be successfully ligated to both functionalities by use of acid-sensitive and/or redox potential-sensitive bonds for delivery across the blood brain barrier and delivery of a payload to brain tissue.

### CROSS REFERENCE TO RELATED APPLICATION

This application is a divisional application of U.S. application Ser. No. 15/164,994, entitled “Dual Responsive Brain Targeted Nanoparticles and Their Applications,” having a filing date of May 26, 2016, which claims filing benefit of U.S. Provisional Patent Application Ser. No. 62/167,563 entitled “Methods of Preparing Brain Targeted nanoparticles and their Application,” having a filing date of May 28, 2015, both of which are incorporated here by reference in their entirety.

### GOVERNMENT SUPPORT CLAUSE

This invention was made with government support under 1P20GM109091-01 awarded by NIH. The government has certain rights in the invention.

### BACKGROUND

Most small molecules useful for the treatment of central neural system disease cannot cross the blood brain barrier. As such, delivery of therapeutics for use in the brain poses many challenges. Development of efficient delivery systems for central nervous system drugs is needed.

### SUMMARY

According to one embodiment, disclosed are nanoparticles suitable for delivery of materials across the blood brain barrier. More specifically, the nanoparticles include a biocompatible hydrophilic polymer and two (or more) types of surface ligands that can encourage transport across the blood brain barrier and then be detached from the nanoparticles by acidic pH and/or high redox potential as may be found in the lysosome or following crossing of the blood brain barrier so as to release the payload carried by the nanoparticles. The nanoparticles can also include a biologically active compound such as a drug, e.g., encapsulated in the nanoparticle or attached to the surface of the nanoparticle, for delivery following crossing of the blood brain barrier by the nanoparticles.

Also disclosed are methods of forming the nanoparticles and methods of using the nanoparticles. For instance, the dual responsive nanoparticles can be formed by conjugation of a hydrophilic biocompatible polymer with the two different ligands by formation of acid-sensitive and/or redox potential-sensitive bonds and by forming the polymer as a nanoparticle, for instance by crosslinking the hydrophilic biocompatible polymer. The dual responsive nanoparticles can also be loaded with a biologically active agent for delivery across the blood brain barrier either during or following particle formation.

The dual responsive nanoparticles can be used to deliver a biologically active compound across the blood brain barrier in treatment of a disease such as neurodegenerative disorders, Alzheimer's disease, Parkinson's disease, traumatic brain injury, stroke, Down syndrome, amyotrophic lateral sclerosis, HIV encephalitis, epilepsy, Huntington's disease, multiple sclerosis, focal cerebral ischemia, addiction, obsessive-compulsive disorder, trichotillomania, bipolar disorder, autism, brain tumor, spinal cord injury or tumor, autism, etc.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates the size distribution of nanoparticles as described herein.

FIG. 2 provides representative in vivo images of nanoparticle distribution in mice.

FIG. 3 provides representative images of brain tissue from an in vivo experiment and imaged ex vivo.

FIG. 4 provides fluorescence images of tissues imaged 4 hours post nanoparticle injection and imaged ex vivo. Tissues include brain (A), lung (B), heart (C), kidney (D), liver (E), and spleen (F).

FIG. 5 provides fluorescence images of tissues collected from a lipopolysaccharide-induced brain inflammatory mouse Alzheimer's disease model after receiving treatment as described herein.

FIG. 6 provides fluorescence images of bran sections collected from the Alzheimer's model mice after receiving treatment.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

Chemical elements are discussed in the present disclosure using their common chemical abbreviation, such as commonly found on a periodic table of elements. For example, hydrogen is represented by its common chemical abbreviation H; helium is represented by its common chemical abbreviation He; and so forth.

As used herein, the prefix “nano” refers to the nanometer scale up to about 500 nm. For example, particles having an average diameter on the nanometer scale (e.g., from about 0.1 nm to about 500 nm) are referred to as “nanoparticles.”

As used herein, the term “polymer” generally includes, but is not limited to, homopolymers; copolymers, such as, for example, block, graft, random and alternating copolymers; and terpolymers; and blends and modifications thereof. Furthermore, unless otherwise specifically limited, the term “polymer” shall include all possible geometrical configurations of the material. These configurations include, but are not limited to isotactic, syndiotactic, and random symmetries.

The term “organic” is used herein to refer to a class of chemical compounds that are comprised of carbon atoms. For example, an “organic polymer” is a polymer that includes carbon atoms in the polymer backbone, but may also include other atoms either in the polymer backbone and/or in side chains extending from the polymer backbone (e.g., oxygen, nitrogen, sulfur, etc.).

The “number average molecular weight” (Mn) is readily calculated by one of ordinary skill in the art, and generally refers to the ordinary arithmetic mean or average of the molecular weights of the individual macromolecules. It is determined by measuring the molecular weight of n polymer molecules, summing the weights, and dividing by n, such as represented in the formula:

$${\overline{M}}_{n = \frac{{\sum }_{i} {N}_{i} {M}_{i}}{{\sum }_{i} {N}_{i}}}$$

where Ni is the number of molecules of molecular weight Mi. The number average molecular weight of a polymer can be determined by gel permeation chromatography, and all colligative methods, like vapor pressure osmometry or end-group determination.

The “weight average molecular weight” (Mw) is readily calculated by one of ordinary skill in the art, and generally refers to:

$${\overline{M}}_{w = \frac{{\sum }_{i} {N}_{i} {M}_{i}^{2}}{{\sum }_{i} {N}_{i} {M}_{i}}}$$

where Ni is the number of molecules of molecular weight Mi. The weight average molecular weight can be determined by light scattering, small angle neutron scattering (SANS), X-ray scattering, gel permeation chromatography, and sedimentation velocity.

The polydispersity index (PDI) is a measure of the distribution of molecular mass in a given polymer sample. The PDI calculated is the weight average molecular weight divided by the number average molecular weight. It indicates the distribution of individual molecular masses in a batch of polymers. The PDI has a value equal to or greater than 1, but as the polymer chains approach uniform chain length, the PDI approaches unity (i.e., 1).

The preparation of dual functionalized nanoparticles is generally provided along with their application. Beneficially, the dual functionalized nanoparticles provide dual targeting to the blood brain barrier and can effectively pass the blood brain barrier and deliver a payload at nervous system tissue by taking advantage of the physiological characteristics of the blood brain barrier and nervous system tissue. In particular, the nanoparticles can be functionalized with ligands that include blood brain barrier transporters that can effectively carry the nanoparticles across the blood brain barrier. The ligands can be bonded to the nanoparticle by environmentally sensitive linkages that can degrade in an environment including acidic pH and/or high redox potential. As such, the nanoparticles can circulate in a subject's system, pass the blood brain barrier through the targeting provided by the two (or more) functional ligands, and then release their payload following entry into the brain environment due to the sensitivity of the ligand attachment bonds to the environment of the blood brain barrier lysosome and/or the nervous system.

The nanoparticles can be prepared from polymeric materials that can be biocompatible, provide long circulation life in a body, and that can be successfully ligated to at least two different ligands via an acidic responsive and/or redox potential-responsive bond formation. For instance, one or both of the functional ligands can be directly or indirectly bonded to the nanoparticle via an acid-sensitive bond such as, without limitation an ester bond, a hydrazone bond, or a cis-aconityl bond. Alternatively, one or both of the functional ligands can be directly or indirectly bonded to the nanoparticle via a redox potential-sensitive bond such as, without limitation, a disulfide bond. Of course, a ligating bond can be both acidic-sensitive and redox potential-sensitive.

As utilized herein, an acidic-sensitive bond can generally refer to a bond that will degrade or otherwise break in an environment of about pH 6.8 or less, for instance about pH 4 to about pH 6.8, and will be more stable in an environment at higher pH (e.g., about 7 or higher). A redox potential-sensitive bond can generally refer to a bond that will degrade in an environment having a redox potential equal to that of a glutathione concentration of from about 0.1 mM to about 10 mM).

Materials that can be ligated to the nanoparticles can include any material that exhibits blood brain barrier transport capabilities. As utilized herein, the term “blood brain barrier transporter” refers to a material that can naturally pass the blood brain barrier. Moreover, a blood brain barrier transporter can encompass a complete transporter as found in nature or a portion or fragment of the natural compound, e.g., only that portion of a transporter that binds a barrier protein as well as synthetic compounds that function as a blood brain barrier transporter. By way of example, and without limitation, suitable transporter functional ligands can include scopine, glutathione, transferrin, melanotransferrin, adenosine, insulin, low-density lipoprotein, leptin, thiamine, rabies virus glycoprotein, TAT peptide, encephalin, angiopep-2, diphtheria toxin, and tetanus toxin. In general, any combination of two (or more) of such transport-capable compounds ligated to a biocompatible nanoparticle as described is encompassed herein.

In one particular embodiment, the Dual Targeted and Dual Responsive Nanoparticles (DTDRN) can be functionalized to include scopine in conjunction with glutathione. Scopine is a tropane alkaloid found in a variety of plants including mandragora root, senecio mikanoides (Delairea odorata), Scopolia carniolica, and Scopolia lurida. Scopine can be prepared by the hydrolysis of scopolamine. Scopine HCl salt is the metabolite of anisodine, which is an α1-adrenergic receptor agonist and has shown activity as a brain targeting moiety (see, e.g., Wang, et al. Bioconjugate Chem., 2014, 25 (11), pp 2046-2054).

Glutathione (GSH) is an endogenous antioxidant. If its concentration in serum is insufficient, some nervous diseases, such as chronic fatigue syndrome, may occur. Research has found that a Na-dependent GSH transporter located on the luminal side of the blood brain barrier manages GSH uptake and a Na-independent GSH transporter located on the luminal side of the blood brain barrier manages efflux of GSH (1996, J. Biol. Chem. 271: 9754-9758). Through conjugation of both scopine and glutathione to a nanoparticle delivery system as disclosed herein, improved delivery of biologically active compounds across the blood brain barrier can be achieved.

In one embodiment, the basic nanoparticle structure of the delivery system can include a copolymer that is the reaction product of a biocompatible hydrophilic polymer and pyridine-2-thiol containing monomer. For instance, the copolymer reaction product can include pyridine-2-thiol side groups pendant to a backbone via a disulfide linkage. The hydrophilic component can form the polymer backbone and/or can form hydrophilic pendant groups off of the backbone. Nanoparticles of the copolymer can be formed via, e.g., a crosslinking reaction in which disulfide bonds of the copolymer are cleaved followed by aerial oxidation. The nanoparticles thus formed can be suitable for safe and effective therapy with the hydrophilic component of the copolymer being at the exterior surface of the particle. The formation of the nanoparticle can endow advantage for CNS therapy. For example, due to the existence of the hydrophilic corona (e.g., polyethylene glycol), the circulation time of the copolymer in a biological system can be greatly extended.

The hydrophilic component of the polymer can be based upon any biocompatible polymer or oligomer capable of reacting with the desired pyridine-2-thiol monomers. By way of example and without limitation, the hydrophilic component can include one or more of polyethylene glycol, poly(N-isopropylacrylamide) (polyNIPAAm), poly(N-(2-hydroxypropyl)methacrylamide) (polyHPMA), poly(acrylic acid) (PAAc), poly(DL-lactic acid-co-glycolic acid) (PLGA), poly(L-histidine), etc.

In one particular embodiment, the copolymer can be formed by reaction of pyridine-2-thiol monomer with poly(ethylene glycol) methacrylate having the general structure:

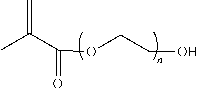

For instance, polyethylene glycol methacrylate used in a formation process can include polymers in which n in the above structure is from about 4 to about 1,000, from about 5 to about 100, or from about 6 to about 20 in some embodiments.

The hydrophilic polymer can react with one or more pyridine-2-thiol monomers to form the polymer that includes the pyridine-2-thiol pendant groups. By way of example, and without limitation, pyridine-2-thiol monomers can include one or more of:

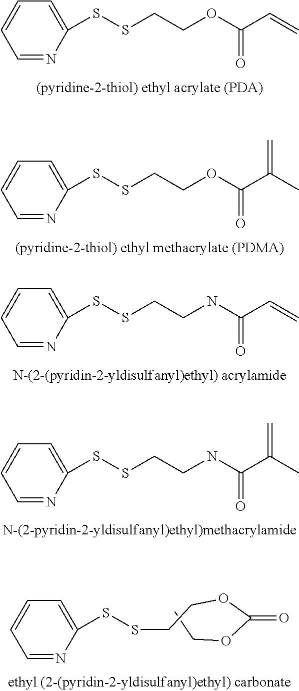 [SMILES: C=CC(=O)OCCSSc1ccccn1 ; C=C(C)C(=O)OCCSSc1ccccn1 ; C=CC(=O)NCCSSc1ccccn1 ; C=C(C)C(=O)NCCSSc1ccccn1 ; CCOC(=O)OCCSSc1ccccn1]

This reaction can be facilitated by any suitable catalyst. For example, the catalyst used in the reaction can be, in particular embodiments, azobisisobutyronitrile (AIBN), benzoyl peroxide, potassium persulfate, or combinations thereof. The polymerization can be free radical polymerization or living radical polymerization including stable free radical mediated polymerization (SFRP), atom transfer radical polymerization (ATRP), reversible addition-fragmentation chain transfer (RAFT) polymerization, and iodine-transfer polymerization. The last monomer of the above examples (ethyl (2-(pyridin-2-yldisulfanyl)ethyl) carbonate) can be polymerized using isopropanol as an initiator and Sn(Oct)₂ as a catalyst through ring-opening polymerization.

The polymerization reaction can form a copolymer that includes pyridine-2-thiol-containing units pendant to the backbone of a polymeric component. For instance, in those embodiments in which the pyridine-2-thiol monomer is polymerized with a poly(ethylene glycol)methacrylate, the resulting copolymer can include pendant groups of the pyridine-2-thiol component, e.g., (pyridine-2-thiol)ethyl acrylate groups and pendant groups of the hydrophilic polymer, e.g., (polyethylene glycol) methacrylate groups and can have the following general structure:

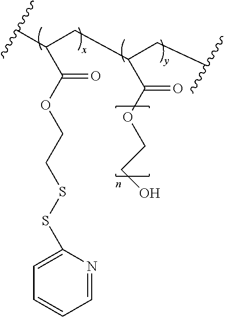

As can be seen, in this particular embodiment, the hydrophilic component of the copolymer will form pendant groups upon the polymerization reaction. In such embodiments, the molar ratio of the pyridine-2-thiol containing repeating units of the polymer to hydrophilic pendant repeating units of the polymer (e.g., the poly(ethylene glycol) methacrylate units) can be from about 100:1 to about 1:100 (the ratio of x to y in the above structure), for instance from about 20:1 to about 1:20 in some embodiments, from about 10:1 to about 1:10 in some embodiments, or about 1:1 in some embodiments.

It should be understood that the hydrophilic polymer that is copolymerized with the pyridine-2-thiol containing monomer need not necessarily form secondary pendant groups as is the case with the poly(ethylene glycol) methacrylate copolymerization process, and in some embodiments, the only pendant groups formed upon reaction of the hydrophilic polymer and the pyridine-2-thiol containing monomers can be the pyridine-2-thiol containing groups.

In addition, although shown as a block copolymer in the above structure, it is to be understood that this representation is simply short-hand for any type of copolymer (e.g., random, block, etc.) that includes repeating units of both the pyridine-2-thiol repeating units and repeating units of the hydrophilic polymer.

The pyridine-2-thiol containing copolymer can generally have a weight average molecular weight from about 1,000 to about 100,000 or from about 5,000 to about 35,000 in some embodiments. In one embodiment, the copolymer can have a PDI of from about 1.05 to about 3 or from about 1.15 to about 1.30 in some embodiments.

In one representative embodiment, the nanoparticles can be prepared by initially forming a copolymer according to reaction of Poly[(2-(pyridin-2-yldisulfanyl) ethyl acrylate-co-[poly(ethylene glycol) (PDA-PEG) followed by functionalization (e.g., amine, acid, imide etc.) via, e.g., thiol-disulfide exchange reaction. Following, the functionalized polymer can be conjugated with one or both of the transporter ligands, e.g., scopine and glutathione, via the formation of bonds that are acid-sensitive and/or redox potential-sensitive bonds. For example, scopine can be conjugated to an acid-functionalized polymer to form an ester link between the polymer and the scopine ligand, while glutathione can be conjugated to a maleimide-functionalized polymer via a sulfur linkage and then conjugated to a nanoparticle component via carbodiimide chemistry to form an ester link between the nanoparticle component and the glutathione ligand. Therefore, both scopine and glutathione are indirectly conjugated to the nanoparticle through PDA segments which contain both ester bonds and disulfide bonds.

The particle form of the delivery system can be provided via crosslinking of the polymeric component. For instance, a PDA-PEG polymer can be subjected to disulfide bond cleavage followed by oxidation to crosslink the polymers and form a nanoparticle. In addition, the functional ligands can be conjugated to the nanoparticles either prior to or following crosslinking and particulate formation. For instance, one or both of the functional ligands can be surface conjugated to the nanoparticles following crosslinking and particle formation to form the delivery system that can facilitate nanoparticle penetration through the blood brain barrier.

The dual targeted nanoparticles thus formed are labile in environments with low pH and/or high redox potential such as the brain (e.g., pH 6.5 and GSH 2.7 mM), which makes the carriers ideal for brain targeted delivery. Due to the unique dual targeted and dual responsive properties provided in certain embodiments, the disclosed systems can serve as a one-way shuttle for the delivery of drugs specifically to the brain.

The nanoparticle delivery system can be responsive to acidic pH and/or high glutathione environment, and the pH in the brain tissue is low and the GSH level is high, which makes the nanoparticle delivery system an ideal tool for brain targeted delivery. The payload (i.e., the drug compound to be delivered to the brain) can be encapsulated into the nanoparticle by hydrophobic interaction or chemically conjugated to the surface through, e.g., —S—S—, —CONH—, or —COO— bonds. Examples of biologically active compounds as may be delivered by use of a system can include, without limitation, n-acetyl cysteine, pyrrolidine dithiocarbamate, disulfiram, diethyldithiocarbamate, tangeritin, resveratrol, indometacin, paclitaxel, doxorubicin, temozolomide, curcumin, carboplatin, carmustine, cisplatin, cyclophosphamide, etoposide, irinotecan, lomustine, methotrexate, procarbazine, vincristine, sulindac, etc., as well as combinations of active agent.

The delivery system can be beneficial in treatment of a wide variety of CNS-related disease states including, without limitation, Alzheimer's disease, Parkinson's disease, traumatic brain injury, stroke, Down syndrome, amyotrophic lateral sclerosis, HIV encephalitis, epilepsy, Huntington's disease, multiple sclerosis, focal cerebral ischemia, addiction (e.g., nicotine, controlled substances, alcohol, gambling, etc.), obsessive-compulsive disorder (e.g., nail biting and skin picking), trichotillomania, schizophrenia, bipolar disorder, autism, brain tumor, spinal cord injury or tumor, etc.

The present invention may be better understood with reference to the Examples set forth below.

### Example 1

### Methods:

### 1. Synthesis of PDA-PEG Polymer

PDA-PEG polymer was synthesized by free radical polymerization as per published methods. (Bahadur K. C, R.; Xu, P. Advanced Materials 2012, 24, (48), 6479-6483.) Briefly, 2-(pyridin-2-yldisulfanyl)ethyl acrylate (PDA) (241.3 mg, 1 mmol), and polyethylene glycol (PEG, MW 360 Da, 360 mg, 1 mmol) were dissolved in 10 mL degassed anisole. 2,2-azobisisobutyronitrile (AIBN, 14 mg, 0.085 mmol) in 1 mL degassed anisole was then added dropwise, and the reaction mixture was stirred for 24 hours at 65° C. The final product was precipitated (3×) in ice cold ether and dried for 48 hours in vacuum.

### 2. Synthesis of Functionalized PDA-PEG Polymer

COOH-PDA-PEG polymer was prepared by thiol disulfide exchange reaction between 0.75 mg mercaptopropionic acid and 20 mg of polymer in 1 ml of a dichloromethane/methanol mixture [1:1 (v/v)] for 3 h at room temperature. The product was precipitated (3×) in ice cold ether and dried for 48 hours under vacuum. For Scopine conjugation, COOH-PDA-PEG (20 mg), scopine (1.38 mg, 8.88 μmol), and DMAP (0.543 mg, 4.44 μmol) were dissolved in 10 ml anhydrous dichloromethane. Then 10 mL anhydrous dichloromethane containing DCC (1.83 mg, 8.88 μmol) was added drop wise. The reaction mixture was stirred overnight at room temperature and then dialyzed through dialysis tube (MWCO 8 kDa) against DMSO.

NH₂-PDA-PEG polymer was prepared by thiol disulfide exchange reaction between 0.4 mg cysteamine and 20 mg of polymer in 1 ml of DMSO for 3 h at room temperature. For Cy7 conjugation, NH₂-PDA-PEG (20 mg) and Cyanine7 NHS ester (0.73 mg) in 1 ml DMSO were reacted overnight and then dialyzed against DMSO.

For the synthesis of GSH-PEG-NH₂ polymer, glutathione (GSH, 10 mg) was first reacted with Maleimide-PEG-NH₂ (0.9 mg, MW 3.4 kDa) in 1 ml PBS overnight under stirring conditions and then dialyzed through dialysis tube (MWCO 1 kDa) against ddH₂O and freeze dried to yield GSH-PEG-NH₂. The 100% consumption of Maleimide double bond was proved by NMR.

3. Nanoparticle Fabrication (Control NP, Scopine NP, GSH NP, and Scopine/GSH NP)

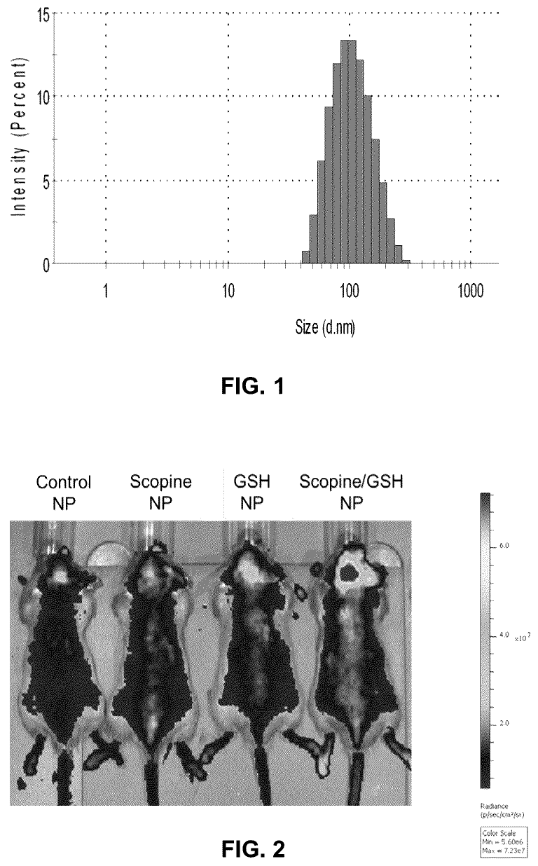

Nanoparticles were prepared by crosslinking reaction of polymer (PDA-PEG) via disulfide bonds cleavage followed by aerial oxidation. Briefly, tris(2-carboxyethyl)phosphine (TCEP, 0.126 mg) in 20 μl DMSO were added in each different formulation (mixture of polymers, Table 1) in total volume of 0.5 ml DMSO and vortexed vigorously. Then, this mixture was added in 5 ml ddH₂O under stirring conditions for 4 hours at room temperature. The final solution was loaded into dialysis bag (MWCO: 1000 Da) and dialyzed against PBS 7.4 for 24 h (1 L×3 times). FIG. 1 presents the size distribution of the PDA-PEG control nanoparticles formed.

TABLE 1
The formulation of polymers for the fabrication of
control NP, scopine NP, GSH NP, and Scopine/GSH NP)

| | COOH-PDA- | | Scopine-PDA- | |
|---|---|---|---|---|
| Formulation | PEG | | PEG | Cy7-PDA-PEG |
| 1. Control NP | 3.5 | mg | — | 1.5 mg |
| 2. Scopine NP | 1.75 | mg | 1.75 mg | 1.5 mg |
| 3. GSH NP | 3.5 | mg | — | 1.5 mg |
| 4. Scopine/GSH NP | 1.75 | mg | 1.75 mg | 1.5 mg |

For the preparation of GSH NP and scopine/GSH NP, the resulting nanoparticle prepared above was further surface modified with GSH by reacting with GSH-PEG-NH₂. GSH-PEG-NH₂ (2.28 mg), N-hydroxysuccinimide (NHS, 0.353 mg) and ethyl(dimethylaminopropyl) carbodiimide (EDC, 0.589 mg) were added in 1 ml nanoparticle dispersion (10 mg/ml) and the mixture was left for overnight reaction at 4° C. followed by dialysis. Finally, the nanoparticles were filtered through 0.45 μm syringe filter and stored at 4° C.

### 4. In Vivo Experiments

Animal studies were conducted under a protocol approved by the University of South Carolina Institutional Animal Care and Use Committee. C57BL/6 mice (6-8 weeks old) were purchased from Jackson laboratory. Fluorescence imaging studies were carried out 0.5, 1 and 4 Hours post i.v. injection (retro-orbital injection of the venous sinus), using the IVIS® Spectrum (Caliper Life Sciences). The mice were anesthetized using isoflurane and transferred to the IVIS instrument to collect full body in vivo images (Ex. 710 nm and Em. 780 nm).

FIG. 2 shows the in vivo images of nanoparticle distribution in the mice. As shown, the GHS NPs and the Scopine/GSH NPs had significantly higher concentration within the brain than the control NPs or scopine NPs.

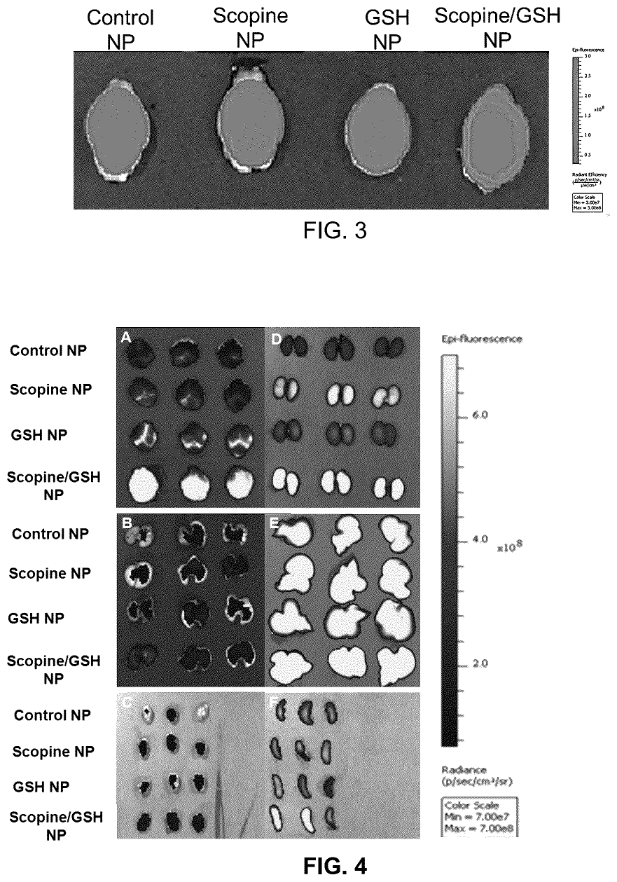

Mice were sacrificed 4 hours post injection and the tissues including brain, spleen, heart, liver, lung, and kidneys were harvested and imaged ex vivo. FIG. 3 shows the brain tissue and FIG. 4 shows the other tissues for the in vivo experiment imaged ex vivo. Here, it is clear that the scopine/GSH NPs had significantly higher concentration within the brain than the other samples.

### Example 2

### Fabrication of N-Acetyl Cysteine (NAC) Loaded DTDRN.

To prepare NAC-loaded dual targeting dual responsive nanoparticles, (NAC-DTDRN), PDA-PEG polymers formed as described above in Example 1 were reacted with NAC through thiol-disulfide exchange reaction to consume 20% PDA groups in DMSO and to yield NAC-PDA-PEG. The resulting polymer was fabricated into NAC-loaded nanoparticles through TCEP initiated crosslinking as described in Example 1. NAC conjugation to PDA-PEG polymer was confirmed by HPLC method with a Waters model 2695 attached to a Waters 2996 photodiode array detector and C18 column using acetonitrile-water (both 0.14% TFA by weight) as the mobile phase. Gradient method was used for analysis from (100:0; water:acetonitrile) to (60:40) in 25 min followed by returning to initial conditions in 5 min at the flow rate of 1 ml/min. The eluted samples were detected at 210 nm. The conjugation efficiency was 99% and the loading content of the nanogel was 5.2%.

### Protection Effect of NAC-DTDRN in a Lipopolysaccharide (LPS) Induced Brain Inflammatory Mouse Alzheimer's Disease Model.

Twelve C57BL/6 mice were divided into 4 groups (n=3). Nine of these mice were injected i.p. with LPS for 10 consecutive days at a dose of 250 μg/kg and the other three mice were injected with PBS i.p. At days 8, 9, and 10 of LPS treatment, each of those three groups were injected i.v. with PBS, free NAC, or NAC-DTDRN at the dose of 0.5 mg NAC per mouse while the control groups will be injected with PBS i.v. At the end of the treatments hydroethidine (1 mg/ml in saline containing 1% dimethylsulfoxide) was administered intraperitoneally. One hour later the mice were be sacrificed and all the organs (heart, liver spleen lungs kidneys and brain) were collected and analyzed ex vivo by IVIS imaging system.

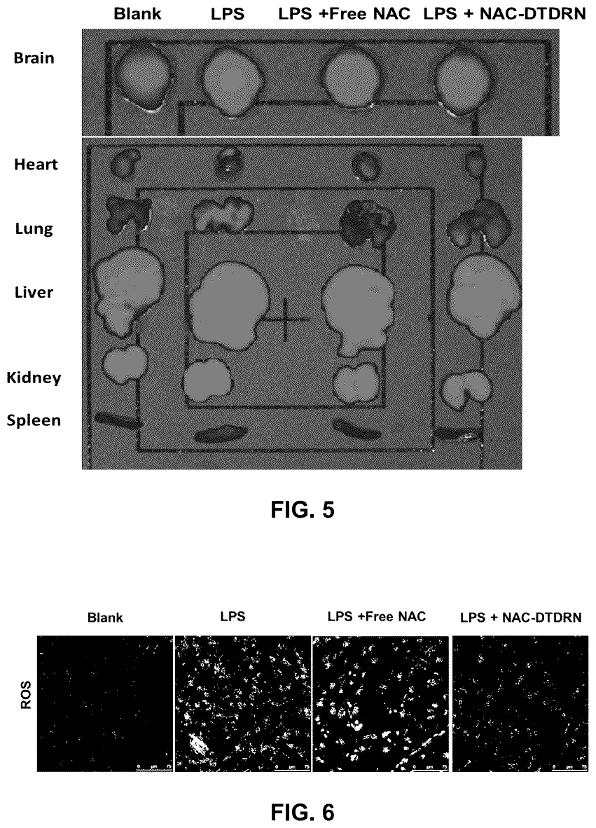

Fluorescence images of tissues collected from the LPS induced inflammatory mouse model after receiving NAC treatment are shown in FIG. 5. As can be seen, the NAC-DTDRN treated mouse exhibited a quenched fluorescence signal in the brain. This clearly illustrates the protection of NAC-DTDRN after it penetrated the blood brain barrier.

Fluorescence images of the brain sections collected from the LPS induced inflammatory mouse model after receiving NAC treatment are shown in FIG. 6. The diminished red fluorescence dots in the brain of NAC-DTDRN treated mouse illustrates that the NAC-DTDRN can effectively reduce brain reactive oxygen species (ROS) level.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole and in part. Furthermore, those of ordinary skill in the art will appreciate that the foregoing description is by way of example only, and is not intended to limit the invention so further described in the appended claims.

What is claimed:

1. A method for delivering a biologically active agent across the blood brain barrier of a subject diagnosed with Alzheimer's Disease, the method comprising:
systemically delivering a nanoparticle to the subject via intravenous or intraperitoneal injection, the nanoparticle including a core that comprises a first (pyridine-2-thiol)-co-poly(ethylene glycol) copolymer that includes a biologically active compound for use in treatment of Alzheimer's Disease conjugated to the first copolymer via substitution at the pyridine-2-thiol pendant groups of the first copolymer, a second (pyridine-2-thiol)-co-poly(ethylene glycol) copolymer that includes a first blood brain barrier transporter conjugated to the second copolymer via substitution at the pyridine-2-thiol pendant groups of the second copolymer, and a third (pyridine-2-thiol)-co-poly(ethylene glycol) copolymer, the core comprising the first, second and third copolymers crosslinked with one another, the nanoparticle further including a second, different blood brain barrier transporter conjugated to a surface of the nanoparticle via substitution at (pyridine-2-thiol) pendant groups of the third copolymer; wherein
following the systemic delivery of the nanoparticle, the nanoparticle crosses the blood brain barrier; and wherein
following the crossing of the blood brain barrier, disulfide bonds of the first, second and third copolymer are degraded, thereby releasing the biologically active compound from the nanoparticle in the brain of the subject.

2. The method of claim 1, wherein the biologically active agent comprises one or more of n-acetyl cysteine, pyrrolidine dithiocarbamate, diethyldithiocarbamate, resveratrol, indomethacin, curcumin, and sulindac.

3. The method of claim 1, wherein the first and second blood brain barrier transporter are independently selected from the group consisting of scopine, glutathione, transferrin, melanotransferrin, adenosine, insulin, low-density lipoprotein, leptin, thiamine, rabies virus glycoprotein, TAT peptide, encephalin, angiopep-2, diphteria toxin, and tetanus toxin.

4. The method of claim 1, wherein the first blood brain barrier transporter is scopine and the second blood brain barrier transporter is glutathione.

5. The method of claim 1, wherein one or more of the first, second, and third copolymers comprise (pyridine-2-thiol) ethyl acrylate-co-poly(ethylene glycol) (PDA-PEG).

6. The method of claim 5, wherein the PDA-PEG comprises 2-(pyridin-2-yldisulfanyl)ethyl acrylate.

7. The method of claim 1, wherein one or more of the first, second, and third copolymers comprise (pyridine-2-thiol) ethyl methacrylate-co-poly(ethylene glycol), N-(2-(pyridin-2-yldisulfanyl)ethyl) acrylamide-co-poly(ethylene glycol), N-(2-(pyridin-2-yldisulfanyl)ethyl)methacrylamide-co-poly(ethylene glycol), or ethyl (2-(pyridin-2-yldisulfanyl)ethyl) carbonate-co-poly(ethylene glycol).
